(19) United States
(12) Patent Application Publication
(10) Pub. No.: US 2012/0213756 A1
(43) Pub. Date: Aug. 23, 2012

(54) NUTRACEUTICAL BEVERAGE

(76) Inventor: Rosemary Petralia, Merrimack, NH (US)

(21) Appl. No.: 13/031,361

(22) Filed: Feb. 21, 2011

(51) Int. Cl. A61K 36/886; A61P 3/02; A61P 39/06; A61K 38/43

(52) U.S. Cl. 424/94.1; 424/744

(57) ABSTRACT

A nutraceutical composition for humans and/or animals containing three main components: water, aloe, and a plant mineral and/or sea mineral complex is provided. The composition provides a 100 percent, all natural, plant derived, completely organic, dietary supplement beverage that is indistinguishable from plain water in clarity, odor, taste and mouthfeel. This formulation supplies a synergy of naturally occurring, bioactive phytonutrients that the body requires, including a combination of macronutrients and micronutrients, e.g.: water, carbohydrates, proteins, lipids, vitamins, minerals, enzymes and antioxidants. This phytosynergistic formulation is designed to fuel cellular hydration, enhance nutrient absorption, promote toxin elimination, improve energy, stamina, digestive health, muscle performance and recovery, and overall health and wellness. This formulation has a negative oxidation reduction potential, which helps to reduce cellular damage and aging caused by free radicals. This formulation has an alkalizing potential to shift the body's pH away from acidification and disease formation.

### TECHNICAL FIELD

This invention relates to beverages. Specifically, this invention relates to a nutraceutical beverage, namely water.

### BACKGROUND

Consuming a sufficient amount of nutrients is extremely important to humans looking to live a healthy life, as well as animals. Isotonic and functional nutraceutical beverages, designed to replace lost fluids, salts, and supply some nutrients, are essentially synthesized, sugar and calorie laden beverages with little to no measurable nutritional value or health benefit. In fact, these drinks containing artificial additives, chemicals, colors, flavors, sugars/sweeteners tout functional benefit, when, in fact, they are nothing more than potentially harmful synthesized solutions with only a limited amount of simple, man made nutrients and little, if any, measurable health advantage. Additionally, many of the ingredients contained in these beverages are required to disguise off-appearances, off-odors, off-tastes, as well as to increase marketability and consumption. The preference, as well as rampant availability, for these synthesized functional beverages over healthy water beverage consumption is negatively impacting public health.

Past nutraceutical beverage formulations have combined water with a variety of ingredients, both natural and artificial. However, these additional ingredients change the appearance, odor, taste, and mouthfeel of the water and require masking to make the beverage more appealing, palatable, and marketable. Furthermore, these synthetic formulations do not confer naturally derived nutrients from plants, which have been found scientifically safe and beneficial for human consumption. Additionally, plant derived nutrients have potential health applications when consumed regularly.

Functional sport/supplement waters on the market today commonly consist of various “types” of treated water combined with either electrolytes and/or trace minerals, as well as targeted vitamins, proteins, carbohydrates, essential nutrients, and herbs. However, these synthesized compositions may also contain potentially toxic metals/minerals from clay sources, unsafe synthetic additives, colors, flavors, sugars/sweeteners, and calories. These synthesized compositions also deliver little or no nutritional or bioactive value and health benefit above that of drinking water. There are also flower essence waters that resemble water, but do not confer any measurable nutritional scope and benefit, nor do they contain any measurable bioactive plant/whole food constituents.

Oxidation reduction potential (ORP) is the ability of any substance to reduce the oxidation of another substance which would be considered an antioxidant. Oxidation reduction potential is a property of water and all liquids. Normal tap water has an ORP of 300 to 400 mV. Thus, its potential for reducing oxidation is non-existent because its ORP is greater than zero. A beverage that has a negative ORP is healthier to consume because it negates oxidation within the body. The lower the ORP, the greater the potential to reverse cellular aging.

Thus, there is a need for a nutraceutical beverage that resembles drinking water in look, odor, taste, and mouthfeel, while also being formulated from 100 percent natural, organic, plant derived, bioactive isolates that have been found to be essential, safe, and beneficial to promoting health and well being.

### SUMMARY

This invention overcomes the disadvantages of the prior art by providing a nutraceutical beverage for humans and/or animals containing three main components: water, aloe, and a plant mineral and/or sea mineral complex. This composition is a 100 percent, all natural, plant derived, completely organic, dietary supplement beverage that is indistinguishable from drinking water in clarity, odor, taste and mouthfeel. The nutraceutical beverage of the present invention is a safe, beneficial supplement beverage that is rich in naturally occurring, bioactive phytonutrients as opposed to the beverages of the prior art that contain empty calories, artificial additives, colors, flavors, sugars/sweeteners, and potentially toxic, clay based minerals. The formulation of the present invention naturally supplies a synergy of naturally balanced, bioactive macronutrients and micronutrients that the body needs. This formulation is designed to fuel cellular hydration, enhance nutrient absorption, promote toxin elimination (detoxification), energy, stamina, digestive health, muscle performance and recovery, and overall health and wellness, especially when consumed regularly. This composition has a negative oxidation reduction potential (ORP), also known as “redox potential” or “antioxidant potential”, which helps to reduce cellular damage and aging caused by free radicals in the body. This formulation also has natural alkalizing potential to shift the body's pH away from acidification and disease formation. This nutraceutical beverage is naturally calorie free due to the organic plant composition and does not require preservatives, additives, colorings, flavorings, or sweeteners. This nutraceutical beverage has the look, smell, taste, and feel of drinking water while conferring significant health benefits when consumed regularly. With this, the nutraceutical water of the present invention has a variety of health applications. This formulation naturally adds a balance of vital nutrients into a consumer's diet.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### DETAILED DESCRIPTION

In one embodiment, the nutraceutical beverage of the present invention contains three main components: water, aloe, and fulvic acid. In an alternative embodiment, other plant minerals may be added to the nutraceutical beverage.

Water

Water is a substance with chemical formula H₂O: one molecule of water has two hydrogen atoms covalently bonded to a single oxygen atom. Water is a tasteless, odorless liquid at standard temperature and pressure. The color of water is, intrinsically, a very slight blue hue, although water appears colorless in small quantities.

Water can dissolve many different substances, giving it varying tastes and odors. The taste of spring water and mineral water, often advertised in marketing of consumer products, derives from the minerals dissolved in it. However, pure H₂O is tasteless and odorless. The advertised purity of spring and mineral water refers to absence of toxins, pollutants and microbes.

The human body is anywhere from 55% to 78% water depending on body size. To function properly, the body requires between one and seven liters of water per day to avoid dehydration; the precise amount depends on the level of activity, temperature, humidity, and other factors. Alternatively, the average daily requirement for water is 35 ml/kg of body weight. Most of this is ingested through foods or beverages other than drinking water alone. It is not clear how much water intake is needed by healthy people, though most advocates agree that 6-7 glasses of water (approximately 2 liters) daily is the minimum to maintain proper hydration. Medical literature favors a lower consumption, typically 1 liter of water for an average male, excluding extra requirements due to fluid loss from exercise or warm weather. In warm, humid weather and while exercising, it is dangerous to drink too little water. People can drink far more water than necessary while exercising, however, putting them at risk of water intoxication (hyperhydration), which can be fatal.

Water plays important roles in the functioning of circulatory, respiratory, excretory and nervous systems, along with metabolic reactions, maintenance of skin components and prevention of a number of disorders. Water has one of the most important roles to play in maintaining a healthy body and some of its benefits include:

pH balance: Human body maintains a pH range of 7.35-7.45 for proper physiological processes. An acidic shift may lead to sickness and inability of the body to assimilate vitamins and minerals. Thus, it should be slightly above the value of 7, which is favorable to body's oxygen uptake, higher energy levels, and better immune response to diseases. This is attainable by consuming copious amounts of water.

Body temperature: Much of the human body is made up of water and this fluid is responsible for regulation of body temperature. The human body perspires to dissipate excess body heat, especially in response to conditions such as warm temperatures and overexertion. This homeostatic property has been attributed to water.

Metabolism: Water is the carrier of oxygen, nutrients, hormones, etc. to body parts and provides medium for removal of toxins, dead cells, waste material, etc. The proteins and enzymes involved in various fundamental processes also require water for their proper functioning.

Breathing: Water plays its role in distributing oxygen all over the human body while collecting the carbon dioxide from all these parts by dissolving these gases.

Constipation, Heartburn, Migraine, Gastritis and Ulcers: A number of disorders are related with water deficiency. Constipation, heartburn, migraine, gastritis and ulcers can be prevented by drinking modest amounts of water routinely.

Kidney stones: Studies have shown that 2 liters or 10 glasses must be consumed each day to prevent kidney stone formulation. Water is the best solvent and this property does not allow the salts and minerals to accumulate to form stones. The salts reaching the kidneys become diluted and eliminated in urine.

Cardiovascular disease: Water can prevent cardiovascular disorders to a large extent. It maintains proper viscosity of blood and plasma and fibrinogen distribution and therefore adequate hydration of vascular components is advisable.

Rheumatoid arthritis: Joints in human body are prone to continuous friction and wear and tear. Joint function may be maintained by adequate water intake. Decrease in water consumption may lead to rheumatoid arthritis.

Backache: The back portion of our body rests on the spinal cord. The spinal disc core is made up of large volume of water and dehydration leads to back pain in such individuals.

Osteoporosis: According to research, individuals consuming adequate amounts of water have reduced risks to osteoporosis and hip fractures.

Pregnant women and Nursing mothers: A pregnant women carries a new life in her womb that develops from her. The nutrients and other factors needed for its growth can be carried from mother to the fetus via blood which in turn needs water. Constipation, bladder infection, and hemorrhoids are common during pregnancy and these can be avoided by drinking copious amounts of water during this period.

Kidney function: The excretory material in the form of urea is highly toxic to body tissues and must be diluted before their passage. This is again done by the “universal solvent” water.

Skin tone: Skin may get affected by frost bite and blisters. It has been established that proper water levels in the body prevents the body from dehydration. This in turn maintains the turgidity of skin. A number of skin problems can be prevented by consuming adequate amounts of water.

In the present invention, the nutraceutical beverage may contain from 4 ounces to 32 ounces of water.

Aloe

Aloe is a genus containing about four hundred species of flowering succulent plants. The most common and well known of these is aloe vera. The components of aloe vera can be divided into the following groups:

Vitamins: It is rich in all vitamins excluding Vitamin D, especially the antioxidant vitamins A (beta-carotene), C and E and even contains a trace of vitamin B12, one of the very few plant sources of this vitamin. This is important for vegetarians and vegans.

Enzymes: Several different types of these biochemical catalysts when taken orally aid digestion by breaking down fat and sugars. One in particular, bradykinase, helps to reduce excessive inflammation when applied to the skin topically and therefore reduces pain, whereas others help digest any dead tissues in wounds. Lipases and proteases which break down foods and aid digestion are present.

Minerals: calcium, sodium, potassium, manganese, magnesium, copper, zinc, chromium and the antioxidant selenium. Although minerals and trace elements are only needed in very small quantities, they are essential for the proper functioning of various enzyme systems in different metabolic pathways.

Sugars: These are derived from the mucilage layer of the plant which surrounds the inner gel and are known as mucopolysaccharides, which enhance the immune system and help in detoxification. Aloe vera contains both mono and polysaccharides, but the most important are the long chain sugars involving glucose and mannose or the glucomannans which have already been referred to. These sugars are ingested whole from the gut, not broken down like other sugars, and appear in the bloodstream in exactly the same form. This process is known as pinocytosis. Once in the blood stream they are able to exert their immunoregulating effect. Some of these polysaccharides are not absorbed but stick to certain cells lining the gut and form a barrier preventing absorption of unwanted material, helping to prevent intestinal permeability/“leaky”gut syndrome.

Anthraquinones: There are twelve of these phenolic compounds which are found exclusively in the plant sap. In small quantities, when they do not exert their purgative effect, they aid absorption from the gastrointestinal tract and have antimicrobial and pain killing effects. In some commercial health drinks, the anthraquinones are removed because of the fear of producing abdominal pain or diarrhea, but they are actually beneficial in small amounts. The important ones, aloin and emodin, act as painkillers. They also function as antibacterials and antivirals.

Lignin: This in itself is an inert substance but when included in topical preparations it endows aloe vera with a singular penetrative effect so the other ingredients are absorbed into the skin.

Saponins: These soapy substances form about 3% of aloe vera and are capable of cleansing, having antiseptic properties. These act powerfully as antimicrobials against bacteria, viruses, fungi and yeasts.

Fatty acids: cholesterol, campesterol, beta-sisosterol and lupeol. These four plant steroids are important anti-inflammatory agents.

Salicylic acid: An aspirin-like compound possessing anti-inflammatory and antibacterial properties.

Amino acids: The body needs 22 amino acids, aloe provides 20 of these. More importantly, it provides 7 out of the 8 essential amino acids which the body cannot synthesize.

Aloe vera has health benefits for certain areas of the body, including the skin, digestive tract, and immune system.

Effect on the Gut

Aloe vera has a wonderfully beneficial action on bowel function which results in a smooth and efficient transit of contents, often eradicating inconvenient, colicky pain.

Assists in Healthy Digestion

A healthy digestive tract ensures that nutrients from the food we eat are absorbed into the bloodstream. Aloe vera enables the bowel to absorb these nutrients more efficiently, especially protein.

Effect on Gut Flora

Aloe vera helps regulate the proportion of bacteria and yeasts that inhabit the gut.

Effect on the Skin

Aloe vera provides the essential nutrition to feed the basal cells of the skin.

Increases the Activity of Fibroblasts

Fibroblasts are specialized cells found in the skin and their job is to produce fiber such as collagen and elastin. These fibers give the skin its structure and, of course, make it look plump and elastic. This is fine from a cosmetic point of view, but they are also extremely important in wound healing, as these fibers create a mesh or network over which the new skin cells advance to close the wound. The effect of aloe vera is to stimulate the fibroblasts to reproduce faster and therefore, being more of them, they make more fiber.

Antiviral Activity

Within the mucilage layer of the aloe vera leaf which surrounds the inner gel there is a long chain sugar or polysaccharide. This sugar has the capability of being able to help defend the body against attacks by various viruses.

In the present invention, the nutraceutical beverage may contain from 0.125 ounces to 2 ounces of aloe vera. The amount is dependent upon the source of raw material as the chemical composition varies according to the precise geographical region from which it was obtained.

Fulvic Acid

Fulvic acid, a plant mineral, is one of two classes of natural acidic organic polymer that may be extracted from humus found in soil, sediment, or aquatic environments. This organic matter is soluble in strong acid (pH=1) and has the average chemical formula C₁₃₅H₁₈₂O₉₅N₅S₂. A hydrogen-to-carbon ratio greater than 1:1 indicates less aromatic character (i.e., fewer benzene rings in the structure), while an oxygen-to-carbon ratio greater than 0.5:1 indicates more acidic character than in other organic fractions of humus (for example, humic acid, the other natural acidic organic polymer that can be extracted from humus). Its structure is best characterized as a loose assembly of aromatic organic polymers with many carboxyl groups (COOH) that release hydrogen ions, resulting in species that have electric charges at various sites on the ion. It is especially reactive with metals, forming strong complexes with Fe³⁺, Al³⁺, and Cu²⁺ in particular and leading to their increased solubility in natural waters. Fulvic acid is believed to originate as a product of microbial metabolism, although it is not synthesized as a life-sustaining carbon or energy source.

Fulvic Acid Major Attributes

Powerful Organic Electrolyte

Fulvic acid is an organic natural electrolyte that can balance and energize biological properties it comes into contact with. An electrolyte is a substance that is soluble in water or other appropriate medium that is capable of conducting electrical current. Fulvic acid has proven to be a powerful organic electrolyte, serving to balance cell life. Fulvic acid has the outstanding ability to accomplish this objective in numerous ways.

Promotes Electrochemical Balance as Donor or Receptor

Fulvic acid is available at times as an electron donor and at other times as an electron acceptor, based on the cell's requirements for balance. One of the reactions that occurs is always an oxidation reaction in which the chemical species loses electrons as a donor. The other reaction is a reduction in which the active species gains electrons as an acceptor. A recent study of the binding of a donor molecule to fulvic acid in solution revealed direct evidence for donor-acceptor charge-transfer mechanisms. Trace minerals in the fulvic acid electrolyte could also be beneficial in this process by serving as electrodes.

Powerful Natural Free Radical Scavenger and Antioxidant

Free radicals of fulvic acid behave as electron donors or acceptors, depending upon the need for balance in the situation. Fulvic acid can in the same way take part in oxidation-reduction reactions with transition metals.

Complexes and Dissolves Minerals and Trace Elements

Fulvic acid is especially active in dissolving minerals and metals when in solution with water. The metallic minerals simply dissolve into ionic form, and disappear into the fulvic structure becoming biochemically reactive and mobile. The fulvic acid actually transforms these minerals and metals into elaborate fulvic acid molecular complexes that have vastly different characteristics from their previous metallic mineral form. Fulvic acid is nature's way of “chelating” metallic minerals, turning them into readily absorbable bioavailable forms. Fulvic acid also has the unique ability to weather and dissolve silica that it comes into contact with.

Enhances Nutrients

Fulvic acid enhances the availability of nutrients and makes them more readily absorbable. It also allows minerals to regenerate and prolongs the residence time of essential nutrients. It prepares nutrients to react with cells. It allows nutrients to inter-react with one another, breaking them down into the simplest ionic forms chelated by the fulvic acid electrolyte.

Transports Nutrients

Fulvic acid readily complexes with minerals and metals making them available to plant roots and easily absorbable through cell walls. It makes minerals such as iron, that are not usually very mobile, easily transported through plant structures. Fulvic acids also dissolve and transport vitamins, coenzymes, auxins, hormones, and natural antibiotics that are generally found throughout the soil, making them available. These substances are effective in stimulating even more vigorous and healthy growth. These substances are produced by certain bacteria, fungi, and actinomycetes in decomposing vegetation in the soil It has been determined that known vitamins can be present in healthy soil. Plants manufacture many of their own vitamins, yet these from the soil further supplement the plant. Upon ingestion these nutrients are easily absorbed by animals and humans, due to the fact that they are in the perfect natural plant form as nature intends. Fulvic acid can often transport many times its weight in dissolved minerals and elements.

Catalyzes Enzyme Reactions

Fulvic acid has close association with enzymes. It increases activity of enzymes, and especially influences respiratory catalysts. Fulvic acids increase the activity of several enzymes including alkaline phosphates, transaminase, and invertase.

Increases Assimilation

Fulvic acid metal organic complexes are of a low molecular weight, and because of this they are also of low molecular size, and are capable of a high degree of penetration into cells. Fulvic acid complexes and chelates are able to readily pass through semi-permeable membranes such as cell walls. Yet it is important to note that it has also been determined that fulvic acids not only have the ability to transport nutrients through cell membranes, they also have the ability to sensitize cell membranes and various physiological functions as well.

Stimulates Metabolism

Fulvic acid appears to cause the genetic mechanism of plants to function at a higher level. It has been concluded that any means by which plant cells are exposed to fulvic acid can improve growth. Oxygen is absorbed more intensely in the presence of fulvic acids. Fulvic acid aids in penetrating roots and then quickly transports to the shoots of plants. Fulvic acid relieves oxygen deficiency and increases the vital activity of cells. Fulvic acids change the pattern of the metabolism of carbohydrates, resulting in an accumulation of soluble sugars. These soluble sugars increase the pressure of osmosis inside the cell wall and enable plants to withstand wilting. Fulvic acid enhances growth and may stimulate the immune system.

Detoxifies Pollutants

An important aspect of humic substances is related to their sorptive interaction with environmental chemicals, either before or after they reach concentrations toxic to living organisms. The toxic herbicide known as Paraquat is rapidly detoxified by humic substances (fulvic acids). Fulvic acids have a special function with respect to the demise of organic compounds applied to soil as pesticides. It has been established that fulvic acid is vital in helping to form new species of metal ions, binding with organic pollutants such as pesticides and herbicides, and catalyzing the breakdown of toxic pollutants. Radioactive substances react rapidly with fulvic acid, and only a brief time is required for equilibrium to be reached. All radioactive elements are capable of reacting with fulvic acid and thus forming organometallic complexes of different adsorptive stability and solubility.

Dissolves Silica

Fulvic acids are especially important because of their ability to complex or chelate metal ions and interact with silica. It has been shown that these interactions may increase the concentrations of metal ions and silica found in water solutions to levels that are far in excess of their assumed dissolution ability.

Synthesizes or Transmutates Minerals

Fulvic acid complexes have the ability to bio-react one with another, and also inter-react with cells to synthesize or transmutate new mineral compounds. The transmutation of vegetal silica and magnesium to form calcium in animal and human bones is a typical example of new synthesis of minerals.

Enhances Cell Division and Elongation

Fulvic acid stimulates and balances cells, creating optimum growth and replication conditions.

Enhances the Permeability of Cell Membranes

Fulvic acids act as specific cell sensitizing agents and enhance the permeability of the cell membrane.

Increases Metabolism of Proteins

Fulvic acid intensifies the metabolism of proteins, RNA, and DNA. It has been found that fulvic acid definitely increases DNA contents in cells, and also increases and enhances the rate of RNA syntheses.

Catalyzes Vitamins Within the Cell

Fulvic acid has the ability to complex vitamins into its structure, where they are presented to the cell in combination with complexed minerals. In this perfect natural condition, they are able to be catalyzed and utilized by the cell. In absence of adequate trace minerals, vitamins are unable to perform their proper function.

Chelates all Monovalent and Divalent Elements

Fulvic acid has the power to form stable water soluble complexes with monovalent, divalent, trivalent, and polyvalent metal ions. It can aid the actual movement of metal ions that are normally difficult to mobilize or transport. Fulvic acids are excellent natural chelators and cation exchangers, and are vitally important in the nutrition of cells.

In the present invention, the nutraceutical beverage may contain from 0.125 ounces to 2 ounces of fulvic acid and/or other plant minerals. The amount is dependent upon the source of raw material as the chemical composition varies according to the precise geographical region from which it was obtained.

In another embodiment of the present invention, the nutraceutical beverage may contain sea mineral complexes, such as sea salts and sea vegetables.

Sea Minerals

Sea minerals are nature's perfect balance of macrominerals, microminerals, and ultra trace minerals. They are present in the most readily assimilated and bio-available form known. Sea mineral levels are almost identical to the mineral levels found in human blood serum and are in pH balance very similar to human blood. Sea minerals are free of toxic heavy metals, such as arsenic, cadmium, mercury, lead, radon, ruthenium, and uranium.

Sea Salt

Sea salt is primarily composed of the following ions, listed in order of descending abundance by weight:

| | |
|---|---|
| Chloride (Cl−) | 55.03% |
| Sodium (Na+) | 30.59% |
| Sulfate (SO42−) | 7.68% |
| Magnesium (Mg2+) | 3.68% |
| Calcium (Ca2+) | 1.18% |
| Potassium (K+) | 1.11% |
| Bicarbonate (HCO3−) | 0.41% |
| Bromide (Br−) | 0.19% |
| Borate (BO33−) | 0.08% |
| Strontium (Sr2+) | 0.04% |
| Other ions | 0.01% |

Although the salinity of sea water varies quite a bit worldwide, the relative amounts of the constituent ions remain the same.

Sea salt allows liquids to freely cross body membranes, e.g.: the glomeruli of the kidney or blood vessel walls. Sea salt is necessary for the proper breakdown of plant carbohydrates into usable and assimilable nutrients.

Sea Vegetables

Sea vegetables come in green, brown, red and blue-green algae. A quick profile:

Kelp (laminaria) contains vitamins A, B, E, D and K, is a main source of vitamin C, and rich in minerals. Kelp proteins are comparable in quality to animal proteins. A brown marine plant, kelp contains sodium alginate (algin), an element that helps remove radioactive particles and heavy metals from the body. Algin, carrageenan and agar are kelp gels that rejuvenate gastrointestinal health and aid digestion. Kelp works as a blood purifier, relieves arthritis stiffness, and promotes adrenal, pituitary and thyroid health. Kelp's natural iodine can normalize thyroid-related disorders like overweight and lymph system congestion. It is a demulcent that helps eliminate herpes outbreaks.

Kombu (laminaria digitata, setchelli, horsetail kelp), has a long tradition as a Japanese delicacy with great nutritional healing value. It is a decongestant for excess mucous, and helps normalize blood pressure. Kombu has abundant iodine, carotenes, B, C, D and E vitamins, minerals like calcium, magnesium, potassium, silica, iron and zinc, and the powerful skin healing nutrient germanium. Kombu is a meaty, high-protein seaweed. It is higher in natural mineral salts than most other seaweeds.

Hijiki is a mineral-rich, high-fiber seaweed, with 20% protein, vitamin A, carotenes and calcium. Hijiki has the most calcium of any sea green, 1400 mg per 100 g. of dry weight.

Nori (porphyra, laver) is a red sea plant with a sweet, meaty taste when dried. It contains nearly 50% balanced, assimilable protein, higher than any other sea plant. Nori is rich in all the carotenes, calcium, iodine, iron, and phosphorus.

Arame (Eisenia bycyclis), is one of the ocean's richest sources of iodine. Herbalists use arame to help reduce breast and uterine fibroids, adhesions, and through its fat soluble vitamins and phytohormones, to normalize menopausal symptoms. Arame promotes soft, wrinkle-free skin, enhances glossy hair and prevents its loss.

Sea palm (Postelsia Palmaeformis), American arame, which grows only on the Pacific coast of North America.

Bladderwrack is packed with vitamin K, an excellent adrenal stimulant. It is still used by native Americans in steam baths for arthritis, gout and illness recovery.

Wakame (alaria, undaria) is a high-protein, high calcium seaweed with carotenes, iron and vitamin C. Widely used in the Orient for hair growth and luster, and for skin tone.

Dulse (palmaria palmata), a red sea plant, is rich in iron, protein, and vitamin A. It is a supremely balanced nutrient, with 300 times more iodine and 50 times more iron than wheat. Tests on dulse show activity against the herpes virus. It has purifying and tonic effects on the body, yet its natural, balanced salts nourish as a mineral, without inducing thirst.

Irish moss (chondrus crispus, carrageenan) is full of electrolyte minerals, calcium, magnesium, sodium and potassium. Its mucilaginous compounds help you detoxify, boost metabolism and strengthen hair, skin and nails.

In the present invention, the nutraceutical beverage may contain from about 5 to 20 drops of sea minerals, or approximately ¼ teaspoon or 0.05 mL.

The foregoing has been a detailed description of illustrative embodiments of the invention. Various modifications and additions can be made without departing from the spirit and scope if this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention.

1. A nutraceutical composition comprising:
water, aloe vera, and a plant mineral, wherein the aloe vera and the plant mineral are dissolved in the water.

2. The nutraceutical composition of claim 1 wherein the plant mineral is fulvic acid.

3. The nutraceutical composition of claim 1 wherein the nutraceutical composition comprises about 4 ounces to about 32 ounces of water.

4. The nutraceutical composition of claim 1 wherein the nutraceutical composition comprises about 0.125 ounces to about 2 ounces of aloe vera.

5. The nutraceutical composition of claim 2 wherein the nutraceutical composition comprises about 0.125 ounces to about 2 ounces of fulvic acid.

6. The nutraceutical composition of claim 1 wherein the nutraceutical composition has a higher alkalizing potential than that of a plain water.

7. The nutraceutical composition of claim 1 wherein the nutraceutical composition has a negative oxidation reduction potential than that of a plain water.

8. The nutraceutical composition of claim 1 wherein the nutraceutical composition supplies essential bioactive macronutrients and micronutrients selected from the group consisting of major nutrient classes consisting of water, carbohydrates, proteins, lipids, vitamins, minerals, enzymes, antioxidants, and a combination thereof.

9. The nutraceutical composition of claim 1 wherein the nutraceutical composition is indistinguishable from a plain water beverage in clarity, odor, taste and mouthfeel.

10. A nutraceutical composition comprising:
water, aloe vera, and fulvic acid, wherein the aloe vera and the fulvic acid are dissolved in the water.

11. The nutraceutical composition of claim 10 wherein the nutraceutical composition comprises about 4 ounces to about 32 ounces of water.

12. The nutraceutical composition of claim 10 wherein the nutraceutical composition comprises about 0.125 ounces to about 2 ounces of aloe vera.

13. The nutraceutical composition of claim 10 wherein the nutraceutical composition comprises about 0.125 ounces to about 2 ounces of fulvic acid.

14. The nutraceutical composition of claim 10 wherein the nutraceutical composition has a higher alkalizing potential than that of a plain water.

15. The nutraceutical composition of claim 10 wherein the nutraceutical composition has a negative oxidation reduction potential than that of a plain water.

16. The nutraceutical composition of claim 10 wherein the nutraceutical composition supplies essential bioactive macronutrients and micronutrients selected from the group of major nutrient classes consisting of water, carbohydrates, proteins, lipids, vitamins, minerals, enzymes, antioxidants, and a combination thereof.

17. The nutraceutical composition of claim 10 wherein the nutraceutical composition is indistinguishable from a plain water beverage in clarity, odor, taste and mouthfeel.

18. A nutraceutical composition comprising:
water, aloe vera, and a sea mineral complex, wherein the aloe vera and the sea mineral complex are dissolved in the water.

19. The nutraceutical composition of claim 18 wherein the sea mineral complex is selected from the group consisting of a sea salt, sea vegetables, and a combination thereof.

20. The nutraceutical composition of claim 18 wherein the nutraceutical composition comprises about a ¼ teaspoon of the sea mineral complex.

21. A nutraceutical composition comprising:
water, aloe vera, a plant mineral, and a sea mineral complex, wherein the aloe vera, the plant mineral, and the sea mineral complex are dissolved in the water.
